(19) United States
(12) Patent Application Publication
(10) Pub. No.: US 2018/0325524 A1
(43) Pub. Date: Nov. 15, 2018

(54) CYCLING BALLOON TAMPONADE TECHNOLOGY FOR MANAGING ESOPHAGEAL VARICES

(71) Applicant: University of Florida Research Foundation, Inc., Gainesville, FL (US)

(72) Inventors: Giuseppe MORELLI, Gainesville, FL (US); Shailendra Singh CHAUHAN, Gainesville, FL (US); Haseeb R. JABBAR, Gainesville, FL (US)

(73) Assignee: University of Florida Research Foundation, Inc., Gainesville, FL (US)

(21) Appl. No.: 15/525,869

(22) PCT Filed: Nov. 10, 2015
(86) PCT No.: PCT/US15/59836

§ 371 Date: May 10, 2017

Related U.S. Application Data
(60) Provisional application No. 62/077,502, filed on Nov. 10, 2014.

(51) Int. Cl. A61B 17/12

(52) U.S. Cl. CPC A61B 17/12099; A61B 2017/00818; A61B 17/1204; A61B 17/12136

(57) ABSTRACT

A device for treating bleeding or non-bleeding esophageal varices is provided herein. A tube body having a proximal end and a distal end and configured for placement into a patient's esophagus, a first balloon component associated with the tube body including at least one first pressure sensor associated therewith, a second balloon component associated with the tube body comprising at least one second pressure sensor associated therewith, a first conduit in fluid communication with the first balloon component, and a second conduit in fluid communication with the second balloon component. The device further including a first communication component for transferring information from the at least one first pressure sensor, and a second communication component for transferring information from the at least one second pressure sensor.

### BACKGROUND

Esophageal varices, or severely dilated veins in the lower esophagus, is a complication of portal hypertension, which results in high venous back flow pressure building up in the esophageal region, commonly caused by a stiff, fibrotic, severely diseased condition of the liver known as cirrhosis. Bleeding esophageal varices is a severe complication of this condition that can lead to hemorrhage and death. The main diseases resulting in esophageal varices include:
1—Liver cirrhosis of various origin (e.g. from drugs, viral hepatitis, alcohol liver toxicity etc.); and
2—Liver parasites such as Bilharzias

Due to the abnormally high venous pressure within the esophageal region, submucosal veins in the lower esophagus can become extremely dilated and prone to bleeding and exsanguination. Prior to bleeding, there is an urgent need to treat the non-bleeding esophageal varices to prevent bleeding. When bleeding does occur, there is an emergent need to achieve immediate hemostasis and eliminate recurrence of bleeding which could otherwise lead to death. Presently, esophageal varices are treated by any of the following methods:
1. EGD (Esophagogastroduodenoscopy) upper endoscopy more commonly with ligation/banding and less frequently with sclerotherapy—performed on bleeding and non-bleeding varices by gastroenterologists in developed countries
2. TIPS (Transjugular Intrahepatic Portosystemic Shunt) procedure—performed on bleeding and non-bleeding varices by interventional radiologists in developed countries
3. Balloon Tamponade—performed on bleeding varices though rarely today in developed countries due to current limitations but has ongoing utility, particularly in rural communities and developing countries

Endoscopic ligation is the most effective method to treat bleeding and non-bleeding esophageal varices. The esophageal varices are ensnared with elastic bands to eradicate the varices. Presently, this kind of ligation is performed using an existing devices, which is used by gastroenterology specialists in tertiary centers or major hospitals throughout the world. This device works by pulling the varices through a unit and releasing a rubber band around the varices for effective ligation. This method, though successful, is not easily accessible to patients in rural communities or throughout the developing world.

Endoscopic sclerotherapy has several disadvantages thus, as a result, has fallen out of favor and is used infrequently today. In sclerotherapy, a solution, such as sodium morrhuate or ethanolamine is endoscopically injected into the submucosa around the esophageal varices to cause inflammation and scarring, so as to close off the vein and reduce the likelihood of bleeding. Disadvantages of this method include the following:
1—Repeated injections may create ulcerations or fibrosis which can lead to esophageal strictures.
2—Some esophageal varices can not be managed by this process.

TIPS procedure is a highly successful method of reducing the back flow pressure in a patient with esophageal varices and is performed by interventional radiologist, again in tertiary hospitals or major hospitals throughout the world. However, similar to the above mentioned endoscopic procedures, it is also not easily accessible to patients in rural communities or throughout the developing world.

Due to cost and ease of performance, balloon tamponade technology is a viable solution to treating esophageal varices in these otherwise inaccessible communities. However, as will be further discussed herein, the inventors have identified several drawbacks to the current use of modern balloon tamponade technology in treating esophageal varices.

### BRIEF DESCRIPTION OF THE DRAWINGS

FIG. 1 shows a side view of a device embodiment.

FIG. 2 shows a a device embodiment with first and second balloon components. FIG. 2a shows a side view of the embodiment with possible expansion of the balloon components. FIG. 2b shows a cross-sectional view of the axis h-h. FIG. 2c shows a cross-sectional view of the axis y-y.

FIG. 3 shows a side view of an embodiment that includes pressure sensors.

FIG. 4 shows a side view of an embodiment implementing an endoscope for placement of the device.

### DETAILED DESCRIPTION

Current balloon tamponade technologies on the market include the Sengstaken-Blakemore tube and the Minnesota tube. Both of these forms of tamponade technology have incredible limitations. Of primary concern, they do not enable for cycling between inflation and deflation. As such, this conventional technology is limited by only continuous application of balloon tamponade and the associated risks of esophageal tissue necrosis and resulting perforation. Moreover, current technologies must be placed blindly, and are very difficult to insert and, as such, appropriate positioning is challenging at best.

By temporarily stabilizing an otherwise active or potentially active esophageal variceal bleed, the embodiments disclosed herein provide additional time to transfer the patient to a tertiary hospital or for the necessary resources to arrive at such a facility if an active bleed were to happen in the middle of the night when resources are limited. On a more global level, developing countries could use the technology for the same purpose. Currently, such a patient in a rural American community or in developing country will need to be rushed to a larger facility but would often expire in the process. It is fair to note that the disclosed embodiments will serve to save lives.

According to one embodiment, provided is a balloon tamponade device designed to temporarily stabilize an active or potentially active esophageal variceal bleed. In a specific embodiment, the device optionally includes a bite plate for appropriate positioning at the mouth and appropriate application of longitudinal pressure. The device includes a tube body having a proximal end and a distal end and walls that define a chamber. In a specific embodiment, the tube includes a proximal segment and a distal segment that are optionally having different properties or made of different materials with different properties. The tube body may have a proximal body portion that is configured to predominantly reside in a patient's esophagus and a distal body portion that is configured to predominantly reside in the patient's stomach near the esophageal/stomach junction, or partially in the esophagus. The tube body comprises a mid-chamber balloon component and a distal balloon component. The mid-chamber and distal balloon components may inflate together or independently. Typically, the mid-chamber and distal balloon components are situated on the distal segment.

The mid-chamber balloon and more distal skirt shaped-chamber balloon are positioned so as to abut the esophagus and proximal stomach, respectively. Each balloon component may be connected to adjacent pressure sensors and to pressure tubing exiting the mouth and may be connected to a pressure regulator for proper cycling between balloon inflation and deflation, which serves to avoid unnecessary tissue necrosis of the esophagus and stomach. All such devices may be inserted via a nasogastric tube at the bedside, or esophagogastroduodenoscopy (EGD), with the aim of applying temporary tamponade to stop an active or prevent a potentially active esophageal variceal bleed and to stabilizing an otherwise unstable patient en-route to a tertiary care center or to a time at which more resources at any given institution become available.

It is believed that no current balloon tamponade technology enables for cycling between inflation and deflation. As such, current technology is limited by only continuous application of balloon tamponade and the associated risks of esophageal tissue necrosis and resulting perforation. According to certain embodiments, the cycling of balloon components between inflation and deflation markedly reduces or altogether eliminates such complications and allows for longer periods of use. Additionally, the disclosed balloon tamponade technology can be placed by way of a nasogastric tube allowing for easy placement. Also, current balloon tamponade technology requires that the patient wear a helmet and that the device be connected to the face shield of the helmet for proper positioning and longitudinal pressure. The incorporation of a bite plate as disclosed herein eliminates such need by providing the appropriate positioning and pressure.

Other related issues:
EGD and TIPS procedures in developed countries have minimized use of balloon tamponade.
In rural communities and developing countries, balloon tamponade technology can:
   Stabilize patients before being transferred to a tertiary center
In developed countries, balloon tamponade technology can:
   Allow for better stabilization of patients prior to EGD or TIPS procedures
   Decrease the need for emergent EGD or TIPS procedures in the middle of the night

### Other Advantages of Inventive Embodiments

1) Embodiments disclosed herein can be placed with the assistance of an endoscope or at the bedside with relative ease
2) Direct visualization while placing this device minimizes any potential for complications such as esophageal rupture from inappropriate placement
3) A nasogastric tube for suctioning can be conveniently inserted through the sleeve once the endoscope is removed.
4) This device is easily removed by simply deflating the sleeve
5) The proximal part of the device connects to a bite block instead of a football helmet.
6) The pressure in the balloon chambers is variable and not continuous, thus decreasing the risk of pressure necrosis

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to those skilled in the art to which the invention pertains.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided. It should be borne in mind that all patents, patent applications, patent publications, technical publications, scientific publications, and other references referenced herein are hereby incorporated by reference in this application in order to more fully describe the state of the art to which the present invention pertains.

It should be noted that the terms “may,” “might,” “can,” and “could” are used to indicate alternatives and optional features and only should be construed as a limitation if specifically included in the claims. Claims not including a specific limitation should not be construed to include that limitation.

It should be noted that, unless otherwise specified, the term “or” is used in its nonexclusive form (e.g. “A or B” includes A, B, A and B, or any combination thereof, but it would not have to include all of these possibilities). It should be noted that, unless otherwise specified, “and/or” is used similarly (e.g. “A and/or B” includes A or B or A and B, or any combination thereof, but it would not have to include all of these possibilities). It should be noted that, unless otherwise specified, the term “includes” means “comprises” (e.g. a device that includes or comprises A and B contains A and B but optionally may contain C or additional components other than A and B). It should be noted that, unless otherwise specified, the singular forms “a,” “an,” and “the” refer to one or more than one, unless the context clearly dictates otherwise.

As will be appreciated by one of skill in the art, embodiments of the present invention may be embodied as a device or system comprising a processing module, and/or computer program product comprising at least one program code module.

Accordingly, the present invention may take the form of an entirely hardware embodiment or an embodiment combining software and hardware aspects. Furthermore, the present invention may include a computer program product on a computer-usable storage medium having computer-usable program code means embodied in the medium. Any suitable computer readable medium may be utilized including hard disks, CD-ROMs, DVDs, optical storage devices, or magnetic storage devices.

The term “processing module” may include a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on operational instructions. The processing module may have operationally coupled thereto, or integrated therewith, a memory component. The memory component may be a single memory component or a plurality of memory components. Such a memory component may be a read-only memory (ROM), random access memory (RAM), volatile memory, non-volatile memory, static memory, dynamic memory, flash memory, an erasable programmable read-only memory (EPROM or Flash memory), an optical fiber, and a portable compact disc read-only memory (CD-ROM), a CD ROM, a DVD (digital video disk), and/or any other electronic device that stores digital information.

A “computer” or “computer unit”, as used herein, is intended to include at least one device that comprises at least one processing module (e.g. processor). In a typical embodiment, a computer unit includes at least one processing module, a memory component, and circuitry connecting the at least one processing module and said memory component in a housing. Optionally, the computer unit includes a computer readable medium and circuitry connecting the processing module and computer readable medium. A computer unit is also intended to include two or more computer units hardwired together.

The computer-usable or computer-readable medium may be or include, for example, but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM), a CD ROM, a DVD (digital video disk), or other electronic storage medium.

The term “communicatingly connected” means that one-way or two-way conveyance or communication of information. Typically, information is electronic information that is conveyed through a wired connection or transmitted wirelessly. Two different computer units may be communicatingly connected, a computer unit and a peripheral device and/or components of a computer unit may be communicatingly connected (e.g., a processing module may be communicatingly connected to memory component.) Communicatingly connected may also include conveyance of information via a network (e.g. internet) to a remote computer. Conveyance of information may include transference of an electronic record, such as transfer of an email or email attachment or transfer of a file via a network. Conveyance of information may include transference of a facsimile file to a facsimile computer unit.

Computer program code for carrying out operations of certain embodiments of the present invention may be written in an object oriented and/or conventional procedural programming languages including, but not limited to, Java, Smalltalk, Perl, Python, Ruby, Lisp, PHP, “C”, FORTRAN, or C++. The program code may execute entirely on the user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

Certain embodiments of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer-readable program code modules. These program code modules may be provided to a processing module of a general purpose computer, special purpose computer, embedded processor or other programmable data processing apparatus to produce a machine, such that the program code modules, which execute via the processing module of the computer or other programmable data processing apparatus, create means for implementing the functions specified in the flowchart and/or block diagram block or blocks.

These computer program code modules may also be stored in a memory component or computer-readable memory that can direct a computer or other programmable data processing apparatus to function in a particular manner, such that the program code modules stored in the computer-readable memory produce an article of manufacture.

The computer program code modules may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart and/or block diagram block or blocks.

The term “patient” or “subject” as used herein refers to a human or non-human animal.

According to one embodiment, provided is a device for treating or stabilizing bleeding or non-bleeding esophageal varices that includes a tube body having a proximal end and a distal end and configured for placement into a patient's esophagus. The device may include a first balloon component associated with said tube body that has at least one first pressure sensor associated therewith. The device may include a second balloon component associated with said tube body having at least one second pressure sensor associated therewith. The first and second balloon components may be associated with a first and second conduit, respectively, for carrying air into and out of the balloon components. The term “air” as used herein may include any gas or mixture of gases. Alternatively, the balloon components may be inflated with a fluid. The first and/or second conduits may be integrated with the walls of the tube body for at least a portion of the length of first and second conduits. The conduits may further include an air controller connector located proximate to their proximal end.

Associated with the first and second balloon components can be a first and second single or series of pressure sensors. The first and second single or series of pressure sensors may include a communication component for transferring information from said pressure sensors to a processing module, typically, outside the patient. The communication components may be a wire or a transmitter to wirelessly transmit information.

The tube body typically includes a proximal segment and a distal segment adjacent to the proximal segment, wherein the proximal and distal segments may be comprised of the same or different materials and/or possess the same or different properties. The first balloon component and the second balloon component typically are positioned on the distal segment with the second balloon component being distally positioned relative to the first balloon component.

In a specific embodiment, the second balloon component has a varied expansion where it expands more radially at the distal region than the proximal region when inflated, so as to form a flared longitudinal cross-section. Typically, the first balloon component expands radially in equal proportion, both proximally and distally, when inflated.

The tube body may further comprise a bite block associated with said proximal end of said tube body. As will be described below, the bite block assists with acquiring appropriate positioning and longitudinal pressure of the tube body as well as maintaining these important parameters while in the patient.

According to a further embodiment, provided is a system for treating or stabilizing bleeding or non-bleeding esophageal varices that includes a tube body as described herein. The system may also include an inflation controller that is in fluid communication with the first and second conduits. The system also includes a processing module that is connected with the pressure sensors and the inflation controller. The processing module controls the amount of inflation to the balloon components and controls cycling of the inflation and deflation of the balloon components to avoid prolonged pressure that can otherwise lead to necrosis of the tissue.

The inflation controller may be an air or fluid pump or some other mechanism for delivering air or fluid to the balloon components. For example, the inflation controller may be a chamber of compressed air for fluid. Typically, the inflation controller will have controllable valves that govern the inflow and/or outflow of gas or fluid from the conduit components.

The term “fluid connection” means a connection by which gas and fluid can be transferred.

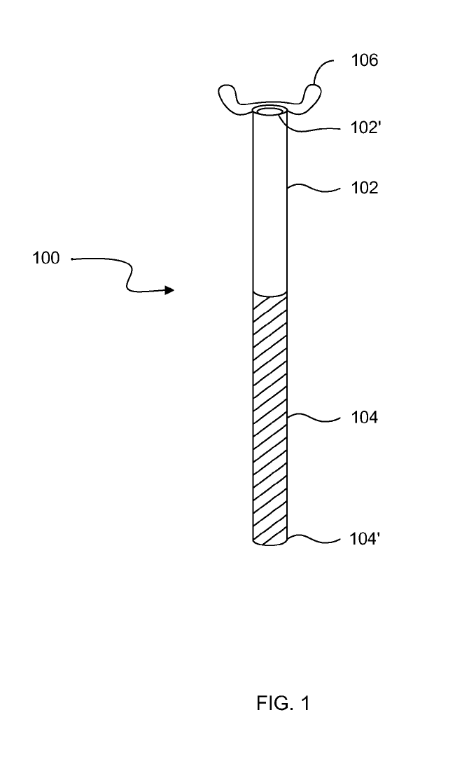

Turning to the drawings, FIG. 1 shows a side view of an embodiment 100 that pertains to a tube 100 that has a proximal segment 102 and a distal segment 104. The tube includes a proximal end 102′ and distal end 104′. Positioned on the proximal end 102′ is a bite block 106. The bite block 106 is arranged to control and stabilize the orientation of the tube 100 in the subject. As will be described infra, the orientation of the tube 100 is important, though not necessarily critical, in executing the balloon tamponade functionality. The proximal segment 102 and the distal segment 104, may be made of similar materials having similar properties. Alternatively, the proximal segment and the distal segment 104 are made of different materials and/or possess different physical properties in terms of flexibility. In a typical embodiment, the distal segment 104 is more flexible than the proximal segment 102. Materials from which the tube may be made include, but are not limited to, rubber, silicone, plastic, fluoropolymers, nylon, thermoplastic elastomers (TPE) (such as PEBA-type TPEs, or polyurethanes), or polyvinyl chloride, or combinations thereof.

In an alternative embodiment, the tube is not a cylinder with a channel therethrough, but can be solid. However, such an arrangement would not allow the pass through of an endoscope or nasogastric tube.

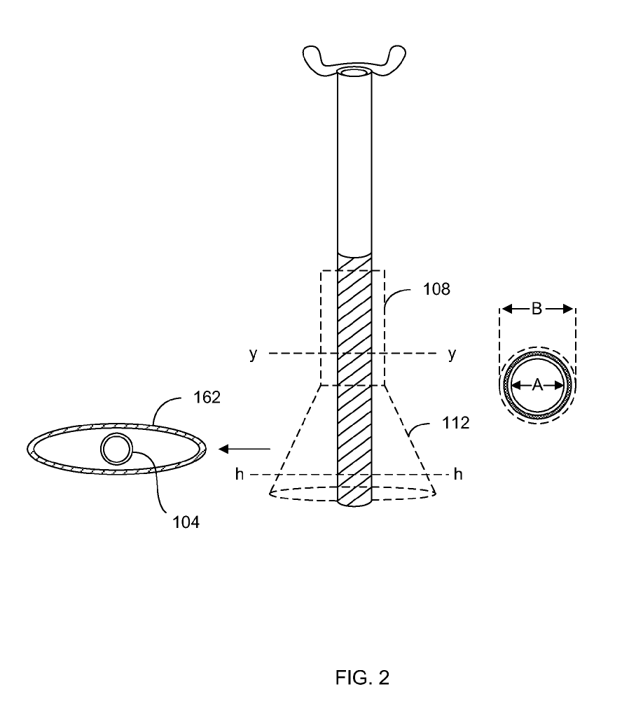

FIG. 2 shows the tube 100 with first balloon component 108 and the second balloon component 112. The first and second balloon components are typically associated with the distal tube segment 104. The first balloon segment expands radially as shown in FIG. 2c, which is a cross-section along the y-y axis. The arrangement of the first balloon component 108 is such that is suitable for abutting against the walls of the esophagus (not shown). FIG. 2c shows the diameter of the first balloon component 108 in a deflated state Second balloon component 112 flares out in a skirt-like fashion. However, as shown in FIG. 2b, the cross-sectional view along the h-h axis, the skirt is eccentric, i.e., having a longitudinal dimension that is bigger than a width dimension, or vice versa. The configuration of the second balloon component 112 is such as to better follow the anatomical contours of the upper stomach.

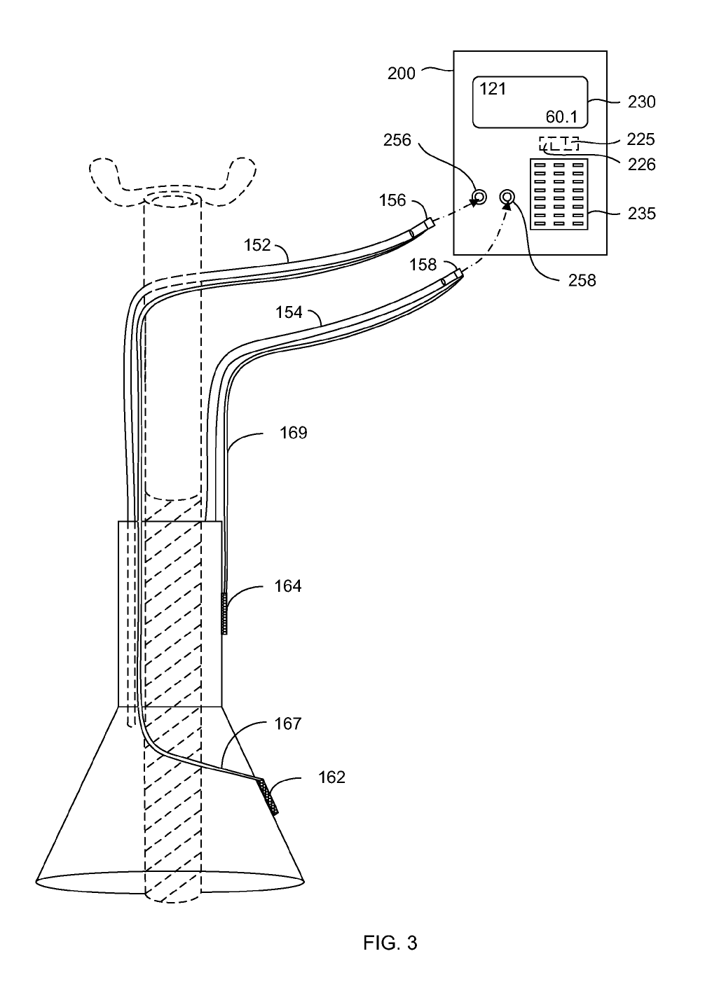

FIG. 3 shows the implementation of conduits with the first and second balloon components 108, 112. Conduit 152 is associated with balloon component 112, and Conduit 154 is associated with balloon component 108. Typically, air or fluid is applied to the conduits to inflate the respective balloon components. Also, shown is the implementation of pressure sensors: pressure sensor 162 associated with the second balloon component 112 and pressure sensor 164 associated with the first balloon component 108. The ends of the conduits 152, 154 include inflation controller connectors 156, 158, respectively, which are adapted for connection to the inflation controller 200. The inflation controller 200 receives information from the pressure sensors, typically via communication components. Communication component 167 is in connection with pressure sensor 162 and communication component 169 is in connection with pressure sensor 164.

The communication components may be electrical wires that may send electrical signal(s) to the inflation controller 200 from the pressure sensors 162, 164, or could even be hollow wires that translate pressure information pneumatically to the inflation controller 200. In this latter alternative embodiment, it is possible that the pressure information could be transferred via the communication components directly from the balloon components without the need for a pressure sensor. It is also noted that the communication components may pertain to transmitters associated with the pressure sensors and that send information wirelessly to the inflation controller.

The inflation controller 200 includes inputs 256, 258 for receiving inflation controller connectors 156, 158, respectively. It is noted that the inputs could be combined into a single input and the connectors could be combined into a harness that plugs into the single input. The inputs 256,258 may also receive communication components 167, 169, respectively, or alternatively, the inflation controller 200 includes separate inputs for the communication components 167, 169. The inflation controller, as shown, may include a display 230 and a input board 235 for programming and actuating control of the inflation controller 200. The inflation controller 200 may include a processing module (not shown) and a memory component (not shown) on which one or more program code modules are stored.

The inflation controller 200 may include, for example, a first program code module for causing said inflation controller 200 to deliver gas and/or fluid to the first and/or second balloon components 108, 112. The inflation controller 200 may further include a second program code module for causing the inflation controller 200 to release gas or fluid from said first and/or second balloon components 108, 112, based on information from pressure sensors 164, 162, respectively. Alternatively, the inflation controller 200 may include a third program code module for causing the inflation controller 200 to cycle gas or fluid in and out of said first and second balloon components 108, 112, based on a predetermined elapsed time of inflation or deflation.

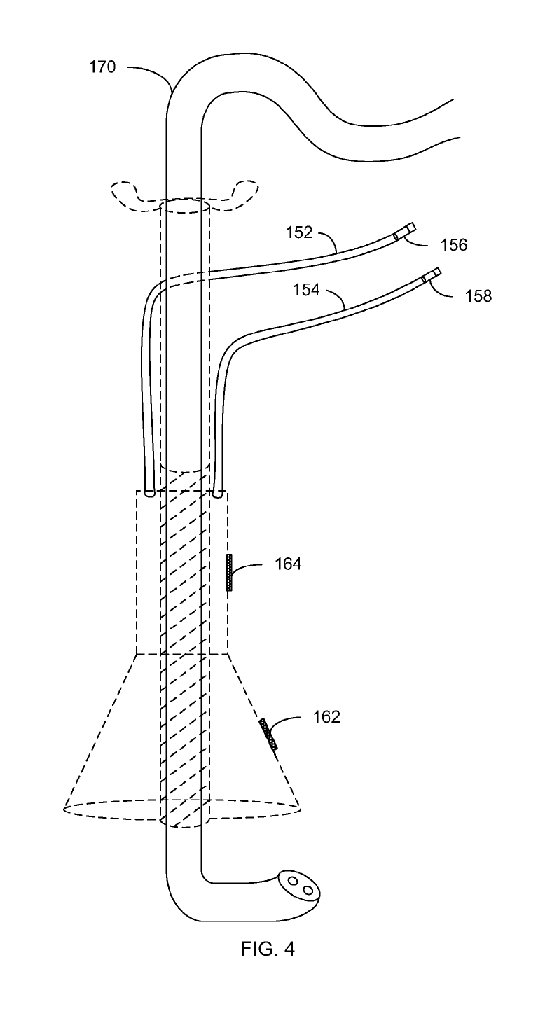

FIG. 4 shows a transparent view of the tube body 100 and balloon components 108, 112 to show the implementation of an endoscope 170 that assists in placement of the embodiment in the esophagus and stomach of the subject. Once in the proper position, the endoscope 170 is removed and a nasogastric tube (not shown) can be placed in the tube body 100 to assist with suctioning of gastric contents.

It should be borne in mind that all patents, patent applications, patent publications, technical publications, scientific publications, and other references referenced herein are hereby incorporated by reference in this application in order to more fully describe the state of the art to which the present invention pertains.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the preceding definitions are provided.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, any means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

While one or more embodiments of the present invention have been shown and described herein, such embodiments are provided by way of example only. Variations, changes and substitutions may be made without departing from the invention herein. Accordingly, it is intended that the invention be limited only by the spirit and scope of the appended claims. The teachings of all references cited herein are incorporated in their entirety to the extent not inconsistent with the teachings herein.

What is claimed is:

1. A device for treating bleeding or non-bleeding esophageal varices comprising;
a tube body comprising a proximal end and a distal end and configured for placement into a patient's esophagus
a first balloon component associated with said tube body comprising at least one first pressure sensor associated therewith;
a second balloon component associated with said tube body comprising at least one second pressure sensor associated therewith;
a first conduit in fluid communication with said first balloon component;
a second conduit in fluid communication with said second balloon component;
a first communication component for transferring information from said at least one first pressure sensor; and
a second communication component for transferring information from said at least one second pressure sensor.

2. The device of claim 1, wherein said tube body comprises a proximal segment and a distal segment adjacent to said proximal segment, wherein said proximal and distal segments are comprised of the same or different materials and/or possess the same or different properties.

3. The device of claim 2, wherein said first balloon component and said second balloon component are positioned on said distal segment with the second balloon component being distally positioned relative to said first balloon component.

4. The device of claim 1, wherein said second balloon component expands more distally than proximally when inflated so as to form a flared longitudinal cross-section.

5. The device of claim 1, wherein said first balloon component expands radially when inflated.

6. The device of claim 1, wherein said first and/or second conduits are integrated with the walls of the tube body for at least a portion of said first and second conduits.

7. The device of claim 1, wherein said first and/or second conduits comprise an inflation controller connector.

8. The device of claim 1, wherein said first and/or second communication component comprises a wire connected to said first and second pressure sensor, respectively.

9. the device of claim 1, wherein said first and/or second communication component comprises a transmitter for transmitting information wirelessly.

9.1 The device of claim 1, further comprising a bite block associated with said proximal end of said tube body.

10. A system for treating bleeding or non-bleeding esophageal varices, said system comprising:
a tube body comprising a proximal end and a distal end and configured for placement into a patient's esophagus
a first balloon component associated with said tube body comprising at least one first pressure sensor associated therewith;
a second balloon component associated with said tube body comprising at least one second pressure sensor associated therewith;
a first conduit in fluid communication with said first balloon component;
a second conduit in fluid communication with said second balloon component;
a first communication component for transferring information from said at least one first pressure sensor; and
a second communication component for transferring information from said at least one second pressure sensor;
an inflation controller in communication with said first and/or second conduits; and
a processing module connected to said first and second communication components and said inflation controller,.

11. The system of claim 10, wherein said inflation controller is an air pump.

12. The system of claim 10, wherein said inflation controller is a pressurized gas or fluid chamber having a valve that controls release of gas or fluid.

13. The system of claim 10, wherein said processing module comprises a memory component with a plurality of program code modules: a first program code module for causing said inflation controller to deliver gas or fluid to said first and/or second balloon components.

14. the system of claim 13, further comprising a second program code module for causing said inflation controller to release gas or fluid from said first and/or second balloon components.

15. The system of claim 10, wherein said tube body comprises an inner channel dimensioned for passing a nasogastric tube therethrough.

16. The system of claim 10, comprising a third program code module for causing said inflation controller to cycle gas or fluid in and out of said first and second balloon components.

17. A method of treating bleeding and non-bleeding esophageal varices in a patient in need thereof, said method comprising:
positioning a tube body in an esophagus and stomach of said patient, wherein said tube body comprises a proximal end and a distal end
a first balloon component associated with said tube body comprising at least one first pressure sensor associated therewith;
a second balloon component associated with said tube body comprising at least one second pressure sensor associated therewith;
a first conduit in fluid communication with said first balloon component;
a second conduit in fluid communication with said second balloon component;
a first communication component for transferring information from said at least one first pressure sensor; and
a second communication component for transferring information from said at least one second pressure sensor;
inflating said first and/or second balloon components such that said first and/or second balloon components abut against an area of need in said patient.

18. The method of claim 17, further comprising deflating said first and/or second balloon components.

19. The method of claim 18, wherein said first and second balloon components are cycled from an inflated and deflated state.

20. The method of claim 19, wherein said cycling is responsive to information from said first and second pressure sensors.
